SEQUENCE LISTING (5 sequences)

SEQ ID NO: 1 - DNA, 20 bp, Artificial Sequence
  ZO-1 Forward Primer
ttttgtcccacttgaatccc

SEQ ID NO: 2 - DNA, 20 bp, Artificial Sequence
  ZO-1 Reverse Primer
agctcttgggtcatgcactt

SEQ ID NO: 3 - DNA, 20 bp, Artificial Sequence
  Occludin Forward Primer
caggttcgcttatcttggga

SEQ ID NO: 4 - DNA, 20 bp, Artificial Sequence
  Occludin Reverse Primer
tccgtagccaaagccactat

SEQ ID NO: 5 - DNA, 1215 bp, Homo sapiens
  gene (1)...(1215): Human Rage Gene
atggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttga

(19) United States
(12) Patent Application Publication
(10) Pub. No.: US 2010/0267031 A1
(43) Pub. Date: Oct. 21, 2010

(54) Method of Screening for RAGE-Amyloid-Beta Peptide Interaction Inhibiting Materials

(76) Inventors: Inhee Mook-Jung, Seoul (KR); Sungmin Son, Seoul (KR)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/682,730

(22) PCT Filed: Oct. 15, 2008
(86) PCT No.: PCT/KR08/06089

§ 371 Date: Jul. 1, 2010

(30) Foreign Application Priority Data
Oct. 15, 2007 (KR) 10-2007-0103702

(51) Int. Cl. C12Q 1/68; C12Q 1/66

(52) U.S. Cl. 435/6; 435/8

(57) ABSTRACT

Provided are a system and a method of screening for RAGE-amyloid beta peptide interaction inhibiting materials using a Transwell plate and a RAGE-expressing cell line.

### TECHNICAL FIELD

The present invention relates to a system of screening for RAGE-amyloid beta peptide interaction inhibiting materials. Also, the present invention is concerned with a screening method using the same.

### BACKGROUND ART

Alzheimer's disease (AD) is the most common form of senile dementia. Now, an estimated 4.5 million people have Alzheimer's disease only in the U.S.A. It is a neurodegenerative disease which is diagnosed in more than 10% of people over 65 years of age worldwide. Alzheimer's disease (AD) is a heterogeneous entity presenting as both a sporadic and a familial disease. A genetic predisposition leads to familial Alzheimer's disease (FAD). Causes of sporadic AD (SAD) are yet unknown. Hereditary Alzheimer's is an uncommon form of Alzheimer's, accounting for only 5-10% of all Alzheimer's disease sufferers. All of the remaining cases are of the sporadic form.

Research indicates that the pathological features of Alzheimer's disease are associated with both amyloid plaques and neurofibrillary tangles in the brain. Amyloid plaques result from the deposition of amyloid-beta peptide (Aβ), and neurofibrillary tangles are pathological protein aggregates formed by hyperphosphorylation of a microtubule-associated protein known as tau, causing it to aggregate in an insoluble form.

Aβ is formed after sequential cleavage of the amyloid-β precursor protein (APP), and is maintained at a constant level in the normal brain. In AD patients, however, excess Aβ buildup is found, forming plaques. These plaques induce neuron loss, causing the impairment of learning and memory.

There exists a control system for maintaining homeostasis of amyloid beta peptide levels in the brain. The balancing function of the system functions is mediated by RAGE (Receptor for Advanced Glycation End products; Zlokovic TRENDS in Neuroscience. 28.2005, D. Stern & Zlokovic et al. Nat. Med. vol 9, 2003), LRP-1(Low density lipoprotein receptor-related protein-1; Zlokovic et al. Neuron, vol 43. 333-344, 2004, D. Stern & Zlokovic et al. Nat. Med., vol 9, 2003) (FIG. 1). RAGE is involved in the influx of amyloid beta peptide from peripheral vessels to the central nervous system by direct interaction with amyloid beta peptide, whereas LRP-1 is involved in the efflux of amyloid beta peptide from the central nervous system to peripheral vessels.

RAGE, which plays an important role in the transport of amyloid beta peptide into the brain, was reported to have an elevated level in patients with Alzheimer's disease (E. Stopa et al. (2006) Acta Neuropathol.). An influx of excess amyloid beta peptide through RAGE activity accelerates the deposition of amyloid beta peptide in the brain, causing the formation of amyloid plaques. Also, RAGE is implicated in various signaling processes through interaction with amyloid beta peptide in addition to the influx of amyloid beta peptide to the brain. Particularly, the interaction of RAGE with amyloid beta peptide was reported to cause the generation of reactive oxygen species (ROS) and inflammation, inducing apoptosis. (Zlokovic et al. (2004) Neuron. vol 43. 333-344) (FIG. 2). Therefore, the inhibition of interaction between RAGE and amyloid beta peptide is expected to block the accumulation of amyloid beta peptide and the downstream signaling thereof, thus exerting a great preventive effect on the progression of Alzheimer's disease.

Initially, RAGE was studied due to the important role thereof in diabetes. Since the disclosure that amyloid beta peptide, a main cause of AD, is a ligand for RAGE, studies on RAGE have been oriented toward correlation with AD. Thus far, efforts to inhibit interaction between RAGE and amyloid beta peptide have been made in two directions toward the use of compounds and the use of soluble RAGE (sRAGE), an isoform of RAGE, which is produced by alternative splicing. As for the compounds, the most advanced molecule is TTP488 of Transtech, which has been studied in the stage 2 trial in patients with Alzheimer's disease, and is currently in a stage 2 study in diabetic nephropathy. This compound is an orally bioavailable small molecule which is reported to reduce the amyloid beta peptide load. Competing with transmembrane RAGE (full-length RAGE) for interaction with amyloid beta peptide, soluble RAGE (sRAGE) is hypothesized to counteract the influx of amyloid beta peptide into the CNS or the signaling by the full-length RAGE. Intensive studies have been conducted to examine the use of sRAGE as a chelator of amyloid beta peptide.

As explained above, most of the studies on materials inhibiting interaction between RAGE and amyloid beta peptide have been directed to the screening of candidates. In contrast, in vitro screening systems by which drug candidates can be examined for effectiveness and are allowed to be applied to clinical stages have been little advanced. In fact, even the leading research groups for RAGE antagonists conduct experiments only to the extent that the signaling processes of new drug candidates are investigated only through RT-PCR or Western blotting. Any system which makes it possible to predict how the candidates act in vivo has not yet been reported.

Leading to the present invention, intensive and thorough research into a system useful in predicting the action modes of the candidates resulted in the finding that a mimic BBB system based on an RAGE-expressing cell line can simulate in vivo BBB and is useful as an effective in vitro system of screening for RAGE-amyloid beta peptide interaction inhibiting materials.

### Disclosure

### Technical Problem

It is therefore an object to provide a system for in vitro screening for a material inhibiting RAGE-amyloid beta peptide interaction.

It is another object of the present invention to provide a method for screening for a material inhibiting RAGE-amyloid beta peptide interaction.

### ADVANTAGEOUS EFFECTS

The screening system according to the present invention is useful in assaying a candidate for inhibitory activity against RAGE-amyloid beta peptide interaction and for passage rate through a paracellular pathway, thus finding a curative for Alzheimer's disease. Further, the system can be applied to the examination of proteins associated with the RAGE signaling cascade, thereby providing information useful in understanding the etiology of Alzheimer's disease.

### DESCRIPTION OF DRAWINGS

FIG. 1 is a schematic view illustrating the exchange of amyloid beta peptide between the brain and the blood by RAGE (Receptor for Advanced Glycation End products) and LRP (Low density Lipoprotein Receptor-related Protein) (Neuron, 43, 605-608, 2004, partially modified).

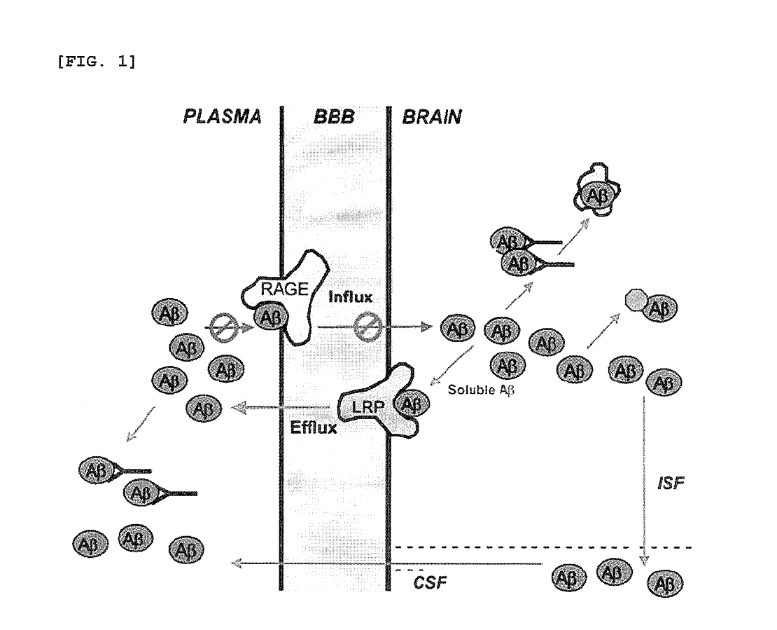

FIG. 2 is a schematic view illustrating a downstream signaling cascade initiated by an RAGE-amyloid beta peptide complex (Stroke, 35(supplI), 2628-2631, 2004).

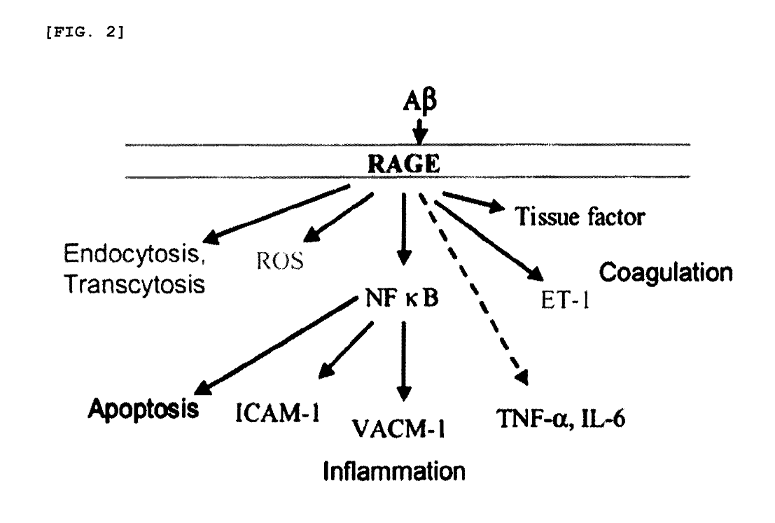

FIG. 3 is schematic views of a system of screening for a candidate inhibitory of RAGE-amyloid beta peptide interaction, showing a structure of an in vitro screening system based on a Transwell plate (a), a change in TEER with the formation of intercellular tight junctions (b), the expression of tight junction proteins through RT-PCR (c), and an increase in the passage rate of a standard material (FD40) due to the interruption of the formation of tight junctions with mannitol.

FIG. 4 is a graph showing materials inhibiting the interaction between RAGE and amyloid beta peptide, identified by the screening system, with a passage rate of 60% for candidate 24, lower than the other candidates 6, 25 and 32.

FIG. 5 is a schematic gene map of a pNFkB-Luciferase vector introduced into CHO, prepared by inserting an enhancer DNA at a position adjacent to NFkB in the MCS (multicloning Site) of a pLuc-MCS vector (Stratagene, Cat. no. 219087).

FIG. 6 is a graph showing candidates for inhibiting interaction between RAGE and amyloid beta peptide, screened by use of the downstream signaling cascade. In a control, the expression level of luciferase conjugated to NFkB is increased by the activation of NFkB in response to RAGE-amyloid beta peptide interaction upon treatment with amyloid beta peptide. In contrast, candidate 24 shows a decreased expression level of the luciferase, demonstrating its inhibitory activity against the RAGE-amyloid beta peptide interaction.

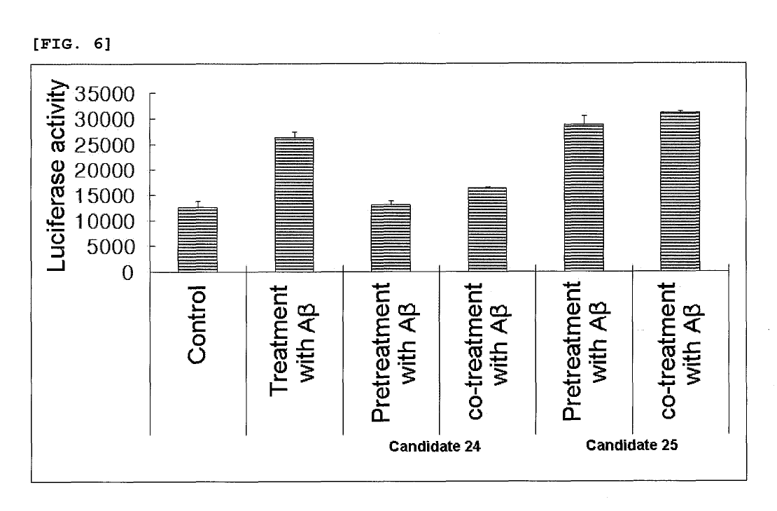

FIG. 7 is a schematic gene map of a hygromycin-selective vector (pcDNA3.1/Hygro(+) vector) (Invitrogen, Cat no. V870-20).

### BEST MODE

In accordance with an aspect thereof, the present invention provides a system for in vitro screening for a material inhibitory of interaction between RAGE and amyloid beta peptide. In greater detail, the system is a mimic blood-brain barrier (BBB) system comprising a cell line which expresses human RAGE.

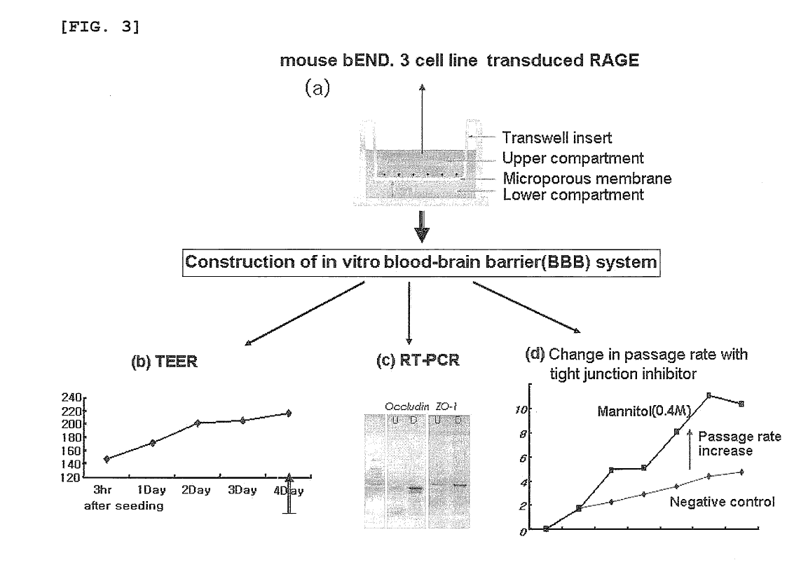

In a preferred embodiment of the present invention, the bEND.3 cell line, which was reported to be suitable for forming a blood brain barrier (BBB) (M. Gumbleton et al. Brain research. vol 990. 95-112, 2003), was used for the construction of the system. The bEND.3 cell line, derived from mouse brain endothelioma, is polyoma middle T-antigen transformed brain endothelial cells. First, bEND.3 cells are cultured at a suitable density in an upper insert of a Transwell plate (Corning, Cat. No. 3495) to form tight junction between cells, as shown in FIG. 3A. Providing a condition similar to the BBB, this intercellular tight junction can serve as a main component of the system of screening for a material inhibiting the interaction of RAGE with amyloid beta peptide. The formation of tight junction between bEND.3 cells can be identified by various assays. For example, TEER (trans-endothelial electric resistance) can be most effectively used to examine the formation of tight junction between endothelial cells. TEER increases with the formation of intercellular tight junction. Thus, comparison with initial TEER values determines the formation of tight junction. On the other hand, ZO-1, occludin and claudins, known as tight junction proteins, increase in expression level as the tight junction forms. On the basis of their mRNA levels which may be analyzed by reverse transcription PCR(RT PCR), the formation of tight junction can be determined. In a more simple method, mannitol, known to interfere with the formation of tight junctions, is applied to the cell culture layer, and then the passage rate of a standard material through the space between cells is compared with that in a negative control to which mannitol is not applied.

In a preferred example of the present invention, the formation of tight junctions between bEND.3 cells was examined by all the above-mentioned three methods. As a result, TEER was observed to increase for four days of culturing (FIG. 3B), and the tight junction proteins ZO1 and Occludin were found to increase in expression level as measured by RT-PCR (FIG. 3C). Also, the formation of tight junctions by bEND.3 cells was ascertained by an increase in the passage of a standard through intercellular spaces after the tight junctions were interrupted by mannitol (FIG. 3C). Based on these results, bEND.3 cells with tight junctions formed therebetween were used in the mimic blood-brain barrier system of the present invention.

The screening system may vary in sensitivity depending on the condition of bEND.3 cells and the introduction efficiency of RAGE gene. In order to exclude the variance, a bEND.3 mutant stain which can overexpress human RAGE is prepared by the introduction of a human RAGE gene with the aid of a hygromycin-selective vector in a more preferred embodiment of the present invention. The cells of interest can be selected by culturing on a plate containing hygromycin.

In accordance with another aspect thereof, the present invention provides a method for screening for a material inhibitory of RAGE-amyloid beta peptide interaction, using the mimic BBB system comprising the human RAGE-overexpressing cell line.

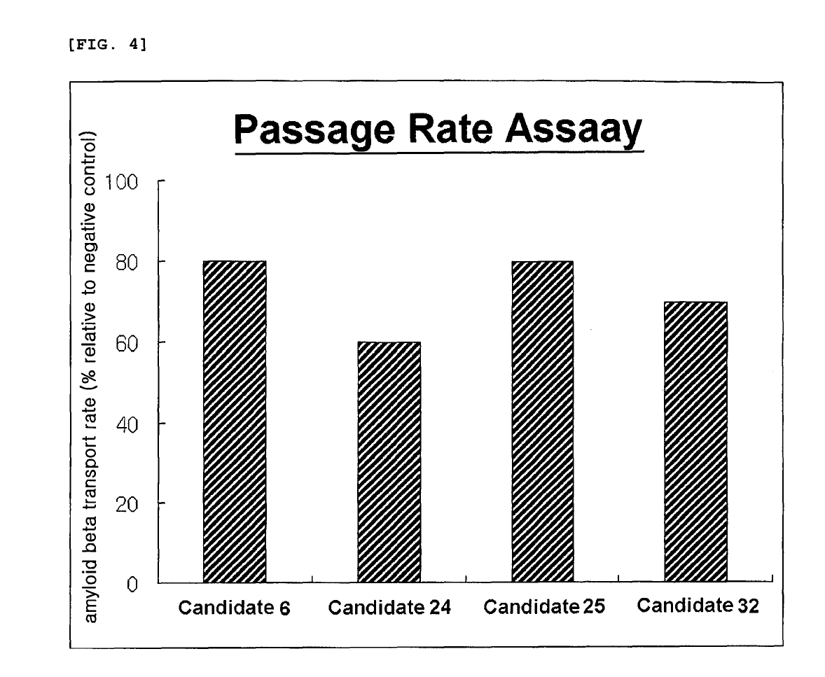

In a preferred example of the present invention, candidates were analyzed for inhibitory activity against RAGE-amlyoid beta peptide interaction using fluorescence (FITC)-labeled amyloid beta peptide. First, the RAGE-overexpressing Bend.3 cell line was prepared and cultured at a concentration in a Transwell plate suitable to form tight junctions therebetween. A candidate was added, along with the fluorescence-labeled amyloid beta peptide, to the upper compartment of the Transwell plate where the cells grew in an attachment manner. Some of the amyloid beta peptide was allowed to migrate into the cells by RAGE while the remainder descended to the lower compartment of the Transwell plate by gravity. The amyloid beta peptide collected in the lower compartment was quantitatively analyzed using a luminescence spectrometer. Comparison with the fluorescence measurement of a negative control in which fluorescence (FITC)-labeled amyloid beta peptide was used without any candidate gave % RAGE inhibition of the candidate (FIG. 4).

In accordance with a further aspect thereof, the present invention provides a system for screening for a material inhibitory of RAGE-amyloid beta peptide interaction using a downstream signaling structure.

As explained above, the RAGE-amyloid beta peptide interaction induces many signaling processes, the earliest of which is NFκB translocation. Thus, the antagonism of a candidate can be determined by comparing NFκB translocation in the system treated with amyloid beta peptide and the candidate with that in the system treated with amyloid beta peptide alone.

In a preferred example of the present invention, expression vectors carrying a human RAGE gene and an NFκB gene, respectively, were co-transfected into CHO (Chinese Hamster Ovary) cells so as to detect the expression of RAGE via NFκB. In greater detail, when a cell line co-transfected with human RAGE and NFκB is treated with amyloid beta peptide, the interaction between RAGE and amyloid beta peptide causes downstream signaling to induce NF-kB translocation, thus resulting in the expression of the luciferase gene of the introduced pNFkB-Luciferase vector within the cell. The expression level can be measured with a luminescence spectrometer.

### MODE FOR INVENTION

A better understanding of the present invention may be obtained through the following examples which are set forth to illustrate, but are not to be construed as the limit of the present invention.

### Example 1

### Establishment of Mimic Blood-Brain Barrier System Using Mouse bEND.3 Cell

1-1. Selection and Culture Condition of Cell Line

The bEND.3 cell line (ATCC CRL2299) was used to construct a mimic blood-brain barrier. Early sub-cultured cells after 5˜10 passages were used. To find a culture condition suitable for forming the same tight junctions as in a blood-brain barrier, the bEND.3 cells were cultured at various cell densities for various culture time periods in 24-well Transwell plates (Corning).

In this regard, the cells were cultured 1×10⁵, 5×10⁴ and 2.5×10⁴ cells per well in 24-well Transwell plates to examine cell status. When cultured at a density of 1×10⁵ cells per well, the cells were too dense to satisfactorily adhere to the bottom of the well and thus to undergo differentiation. Under an optical microscope, the cells were observed to be poor in status. At a density of 2.5×10⁴ cell per well, it took one week or longer for the cells to form tight junctions. Also, the time taken for differentiation was extended. In contrast, tight junctions were formed within a short time at a density of 5×10⁴ cells per well. The cells were stabilized and differentiated for 4 days in a Dulbecco's Modified Eagle's Medium (Hyclone, Salt Lake City, Utah, USA) supplemented with 10% (v/v) fetal bovine serum (FBS, Hyclone, Salt Lake City, Utah, USA) and 1 mg/ml Penicillin/Streptomycin (P/S; Sigma, Saint Louis, Mo., USA). These cells were cultured at 37° C. in a 5.0% CO₂ incubator. 3 hours after seeding the cells started to adhere to the Transwell plates, and the cells were monitored for TEER every day.

The formation of tight junctions was determined by TEER (trans-endothelial electric resistance), RT-PCR, and the permeation of a standard material through the cells.

1-2. TEER (Trans-Endothelial Electric Resistance)

Millicell-ERS (Millipore) was used to measure cell electric resistance. The cells were cultured in 24-well Transwell plates. From 3 hours after seeding, at which time the cells started to adhere to the plates, to Day 4 after the initiation of culturing, the cells were measured for electric resistance at five predetermined time points. First, the electrodes were stabilized and used to measure the voltage of blank wells in which no cells were contained. The measurement of blank wells was called R-blank. Then, all the wells containing the cells therein were measured for voltage and the mean of the measurements was called R-sample. The electric resistance of the cells was obtained by subtracting R-blank from R-sample. As seen in FIG. 3B, the cells were observed to increase in TEER over a period from 3 hours to Day 4 of culturing, demonstrating that stronger intercellular tight junctions were formed with increasing of the culturing period.

1-3. Reverse Transcription PCR(RT-PCR)

The expression of the tight junction peptides ZO-1 and Occludin was determined on an mRNA level. In this regard, the cells were detached from the upper insert of the Transwell plate using trypsin-EDTA on Day 4 after seeding and were washed with PBS. The cells were obtained as a pellet after centrifugation and were used to isolate total RNA therefrom using a TriZol reagent (Invitrogen). cDNA was synthesized from the total RNA using oligo(dT) primers in the presence of a reverse transcriptase (Superscript II reverse transcriptase) and was used as a template for PCR using primer sets for targeting ZO-1 and Occludin.

The results are shown in FIG. 3C. The descriptions for PCR conditions and primers are as follows.

*PCR Conditions

Reverse Transcription—42° C. 90 min, 94° C. 10 min

Polymerase Chain Reaction

cycle 1) 94° C. 5 min (1 cycle)

cycle 2) DNA Denaturation—94° C., 30 sec
DNA Annealing—60° C., 30 sec
DNA Polymerization—72° C., 1 min(30 cycles)

cycle 3) 72° C. 5 min (1 cycle)

Primers

1) ZO-1 (PCR product: 594 base pairs in size)

| | |
|---|---|
| Forward: 5′-TTTTGTCCCACTTGAATCCC-3′ | (SEQ ID NO. 1) |
| Reverse: 5′-AGCTCTTGGGTCATGCACTT-3′ | (SEQ ID NO. 2) |

2) Occludin (PCR product: 406 base pairs in size)

| | |
|---|---|
| Forward: 5′-CAGGTTCGCTTATCTTGGGA-3′ | (SEQ ID NO.3) |
| Reverse: 5′-TCCGTAGCCAAAGCCACTAT-3′ | (SEQ ID NO.4) |

1-4. Comparison of Passage Through Intercellular Space

The cells were seeded on the inserts of 24-well Transwell plates and cultured for 5 days as described above examples. F—Na (10 μM, Sigma, Cat No. 28803), FD4 (1 mM, Sigma, Cat No. FD4), and FD40 (25 μM, Sigma, Cat No. FD40S), all labeled with fluorescence (FITC), were used as standard materials for intercellular passage comparison. Mannitol (0.8 M), known to interfere with the formation of intercellular tight junctions, was added to some wells while other wells were not treated with mannitol. Thereafter, the standard materials were added to the wells, followed by monitoring the fluorescence intensity in the lower compartment of the Transwell plates at regular intervals of 20 min over 120 min. The fluorescence intensity was found to increase with time, but higher intensities were detected in the wells treated with mannitol than in those not treated with mannitol. FIG. 3D shows results of FD40.

F—Na, FD4 and FD40 were 376 Da, 4,000 Da and 40,000 Da in size, respectively. When tight junctions are formed, the materials which can pass between cells are limited by the sizes thereof. Thus, the standard materials may be used as positive or negative controls. For example, when tight junctions are well formed, FD40 differs in passage through intercellular spaces from F—Na and FD4 due to difference in protein size. When the formation of tight junctions was interrupted by mannitol, large FD40 was found to have an intercellular passage degree similar to that of small F—Na and FD4, thus demonstrating the formation of tight junctions.

The results obtained from the experiments of Examples 1-2 to 1-4 identified tight junctions formed between bEND.3 cells and the cells were therefore judged to be suitable for use in the construction of a mimic blood-brain barrier.

### Example 2

### Establishment of Mouse bEND.3 Cell Line Overexpressing Human RAGE

A human RAGE gene (SEQ ID NO. 5) was introduced via a hygromycin-selective vector into bEND.3 cells.

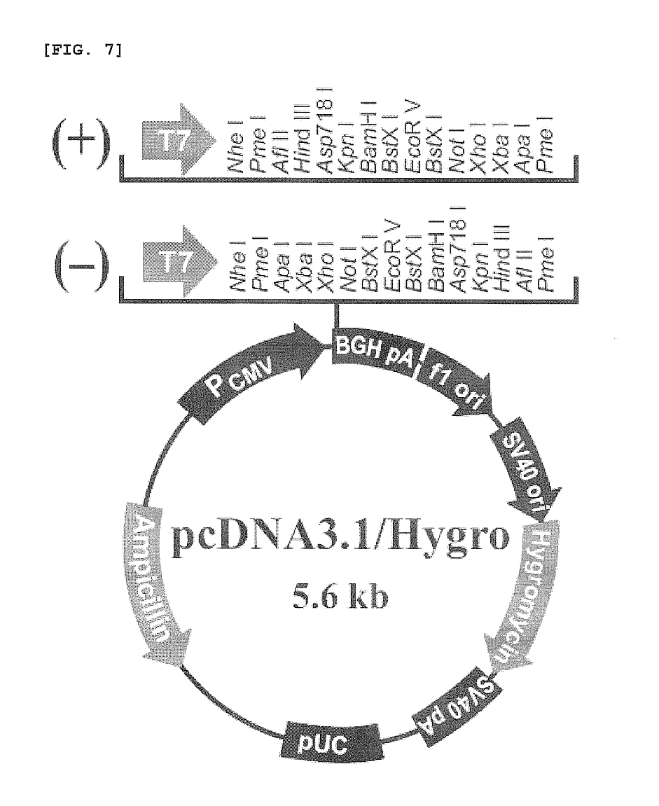

First, the hydromycin-selective vector was the pcDNA3.1/Hygro(+) vector (Invitrogen, Cat no. V870-20) shown in FIG. 7. The human RAGE gene and the pcDNA3.1/Hygro(+) vector were separately digested with the two restriction enzymes Xho1 and Kpn1, followed by ligation to each other in the presence of a ligase. The recombinant human RAGE-pcDNA3.1/Hygro(+) vector thus obtained was transfected into bEND.3 cells with the aid of a Lipofectamin™ LTX reagent (Invitrogen, Cat. No. 15338-100) in combination with a Plus™ reagent (Invitrogen, Cat. No. 11514-015). The bEND.3 cells were cultured on plates containing hygromycin (200 μg/ml, AMRESCO, Cat. No. K547). Colonies formed after incubation for 2˜3 weeks were picked and cultured in broth to establish a cell line expressing the human RAGE gene. After many passages, the cell line was found to stably express the human RAGE gene as assayed by Western blotting analysis.

### Example 3

### Assay for Antagonism Efficiency Against Transport of Amyloid Beta Peptide Using Mimic BBB

Candidates were assayed for inhibitory activity against RAGE by measuring the fluorescence of the fluorescence (FITC)-labeled amyloid beta peptide (beta-Ala-Amyloid beta-Protein, aa 1-42, FITC Conjugated/Tagged, Cat. No. M-2585.1000, Bachem) which passed through paracellular pathway with a luminescence spectrometer.

The human RAGE-overexpressing cell line was cultured at such a concentration in the upper inserts of Transwell plates as to form tight junctions, after which the cells were divided to two groups. One was a negative control to which an FITC-conjugated amyloid beta peptide was added alone. The FITC-conjugatedamyloid beta peptide was also added, but in combination with a candidate, to the other group. The fluorescence of the lower compartment was measured at regular intervals of 20 min over 120 min using a luminescence spectrometer. The two groups were compared with regard to the average of the measurements (FIG. 4). As shown in FIG. 4, candidates 6, 24, 25 and 32 were found to have inhibitory activity against RAGE because the fluorescence-labeled amyloid beta peptide was detected in lower levels in the lower compartment than was the control (with candidate 24 being the highest among them: 60% passage as compared to 100% for the control). In greater detail, first, a Krebs-Ringer bicarbonate buffer (K buffer; Sigma, Cat. No. K4002) was prepared and used as a solution for candidates. Separately, the human RAGE-expressing cells were cultured at a density of 5×10⁴ cells per well for 4 days in the upper compartment of the Transwell plate to allow tight junctions to be formed. Then, the upper and the lower compartment were washed twice with the K buffer. 200 μl of a solution of a candidate in K buffer was carefully poured to the upper compartment while 1 ml of K buffer was added to the lower compartment. At regular intervals of 20 min, 200 μl aliquots of K buffer were collected from the lower compartment over 2 hours. The K buffer samples were placed in a white plate (96 well, SPL, Cat. No. 31196) and measured for fluorescence using a luminescence spectrometer. The passage rate of a candidate was expressed as a percentage of the average of the five measurements relative to the average of a control. As shown in FIG. 4, candidate 24 was found to be more inhibitory of RAGE than candidates 6, 25 and 32 as determined by the lower average fluorescence intensity detected in the lower compartment (60% passage rate of candidate 24 relative to 100% of a control). The other candidates showed about 80% passage rate.

Thereafter, the cells were analyzed for TEER and no changes were found before and after the fluorescence assay, indicating that the increased passage rate was not attributed to the interruption of tight junctions by the toxicity of candidates.

### Example 4

### Construction of System for Screening for RAGE-Amyloid Beta Peptide Interaction Using Downstream Signaling Structure

NFκB translocation was compared between systems treated respectively with amyloid beta peptide alone and in combination with antagonist candidates.

4-1. Establishment of Cell Line Co-Transfected with Human RAGE and NFκB

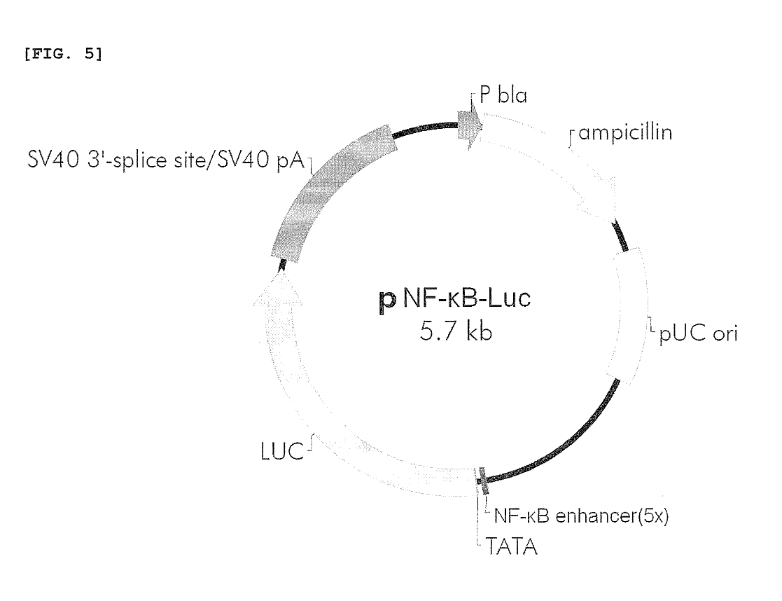

A pNFkB-Luciferase vector (FIG. 5) and a human RAGE gene were cotransfected into CHO (Chinese Hamster Ovary) cells to construct a stable mutant cell line.

To begin with, an enhancer DNA (enhancer base sequence: (TGGGGACTTTCCGC)5), which is effective for the expression of NFκB, was synthesized. This enhancer was inserted into the MCS (multi-cloning site) of the pLuc-MCS vector (Stratagene, Cat. no. 219087). When activated under various stress conditions, NFκB located in the cytosol is translocated into the nucleus. In this MCS, an NFκB gene was cloned at a position adjacent to the enhancer labeled with a fluorescent gene, so that to what extent the cells were stressed could be assessed by measuring the fluorescent intensity. The pNFkB-Luciferase vector thus constructed was introduced into the CHO cell line transformed with RAGE to establish a mutant CHO cell line co-transfected with human RAGE and NFκB. The cotransfection was carried out with the aid of Lipofectamin™ LTX (Invitrogen, Cat. No. 15338-100) in combination with Plus™ (Invitrogen, Cat. No. 11514-015).

The CHO cell line with human RAGE introduced thereinto was established in the same manner as in the mouse bEND.3 cell transformed with human RAGE. As described in Example 2, the human RAGE gene was inserted into the hygromycin-selective vector which was in turn introduced into CHO, followed by selection on plates containing hygromycin. The colonies thus formed were amplified in broth.

4-2. Luciferase Reporter Assay

In the cell line with human RAGE and NFκB co-transfected thereinto, prepared in Example 4-1, the activation of RAGE was determined by measuring the expression of the Luciferase gene conjugated to the RAGE. The cells were washed with PBS and a cell lysis buffer (40 mM tricine (pH 7.8), 50 mM NaCl, 2 mM EDTA, 1 mM MgSO4, 5 mM DTT. 1% Triton X-100 were mixed and five-fold diluted in distilled water) was added in an amount of 50 μL to each well of the 24-well Transwell plates, followed by incubation at room temperature for 15 min to detach the cells. 5˜20 μL of the cell suspension thus formed was well mixed with 100 μL of a Luciferase assay reagent (40 mM tricine(pH 7.8), 0.5 mM ATP, 10 mM MgSO4, 0.5 mM EDTA, 10 mM DTT. 0.5 mM coenzyme A, 0.5 mM luciferin) in a 5-ml round bottom polystyrene tube (BD Falcon), after which a luminometer was used to read entire 24-well Transwell plates in 2 hours with an integration time of 10˜30 sec per well. The control in which amyloid beta peptide was used alone showed a high luciferase expression level due to the interaction between RAGE and amyloid beta peptide. In contrast, when amyloid beta peptide was added in combination with candidate 24, a lower luciferase expression level was measured, indicating that candidate 24 functions to inhibit RAGE-amyloid beta peptide interaction.

Taken together, the data obtained through the assay for antagonism efficiency against amyloid beta peptide transport and the assay using the downstream signaling structure demonstrate that the system based on the mimic BBB in accordance with the present invention can be very useful for screening for RAGE-amyloid beta peptide interaction inhibiting materials.

1. A method of screening for a RAGE-amyloid beta peptide interaction inhibiting material, comprising:
a) preparing a RAGE-expressing cell line;
b) culturing the cell line in such a manner as to form tight junctions between cells in an upper compartment of a Transwell plate;
c) adding an amyloid beta peptide and a candidate for inhibiting RAGE-amyloid beta peptide interaction to the upper compartment of the Transwell plate; and
d) comparing a concentration of the amyloid beta peptide in a lower compartment of the Transwell plate with an amyloid beta peptide concentration obtained in the lower compartment when no candidates are used.

2. The method according to claim 1, wherein the cell line is mouse brain microvascular endothelial cells (bEND.3).

3. The method according to claim 1, wherein the RAGE is human RAGE.

4. The method according to claim 1, wherein the amyloid beta peptide is labeled with a fluorescent material.

5. The method according to claim 4, wherein the fluorescent material is luciferase.

6. A system for screening for RAGE-amyloid beta peptide inhibiting materials, comprising a RAGE-expressing cell line which is cultured in such a manner in a Traswell plate as to form tight junctions between cells.

7. The system according to claim 6, wherein the cell line is bEND.3.

8. The system according to claim 6, wherein the RAGE is human RAGE.

9. A method of screening for an antagonist against RAGE-amyloid beta peptide interaction, comprising contacting amyloid beta peptide and a candidate for inhibiting RAGE-amyloid beta peptide interaction with a cell line co-transfected with recombinant expression vectors respectively carrying RAGE and NFκB, and measuring an expression pattern of NFκB.

10. The method according to claim 9, wherein the recombinant expression vector carrying NFκB comprises a luciferase gene at a position adjacent to the NFκB gene.

11. The method according to 9, wherein the cell line is a CHO (Chinese Hamster Ovary) cell line.
